(19) United States
(12) Patent Application Publication
(10) Pub. No.: US 2009/0326234 A1
(43) Pub. Date: Dec. 31, 2009

(54) (S)-(-)-Amlodipine Camsylate or Hydrate Thereof And Pharmaceutical Composition Comprising Same

(75) Inventors: Jaeheon Lee, Yongin-si (KR); Moon Sub Lee, Osan-si (KR); Weon Ki Yang, Yongin-si (KR); Jaeho Yoo, Yongin-si (KR); Jae-Chul Lee, Suwon-si (KR); Chang-Ju Choi, Seoul (KR); Han Kyong Kim, Yongin-si (KR); Young-Kil Chang, Seoul (KR); Gwansun Lee, Seoul (KR)

Correspondence Address: SUGHRUE MION, PLLC, 2100 PENNSYLVANIA AVENUE, N.W., SUITE 800, WASHINGTON, DC, 20037, US

(73) Assignee: Hanmi Pharm Co., Ltd., Hwaseong-gun (KR)

(21) Appl. No.: 12/374,087

(22) PCT Filed: Jul. 16, 2007
(86) PCT No.: PCT/KR07/03444

§ 371 Date: Jan. 16, 2009

(30) Foreign Application Priority Data
Jul. 21, 2006 (KR) 10-2006-0068401

(51) Int. Cl. C07D 213/80

(52) U.S. Cl. 546/321

(57) ABSTRACT

Disclosed is (S)-(−)-amlodipine camsylate or a hydrate thereof having good photostability and high solubility, and a pharmaceutical composition comprising same, which can be efficiently used in treating cardiovascular diseases.

### FIELD OF THE INVENTION

The present invention relates to an (S)-(−)-amlodipine camsylate or a hydrate thereof which has good photostability and high solubility, and a pharmaceutical composition comprising same.

### BACKGROUND OF THE INVENTION

Amlodipine, a generic name for 3-ethyl-5-methyl-2-(2-aminoethoxy-methyl)-4-(2-chlorophenyl)-6-methyl-1,4-dihydro-3,5-pyridine-dicarboxylate, is a long-term calcium-channel blocker useful for treating cardiovascular diseases such as angina pectoris, hypertension and congestive cardioplegia.

As shown below, amlodipine exists in the form of two enantiomers having a chiral carbon at 4-position.

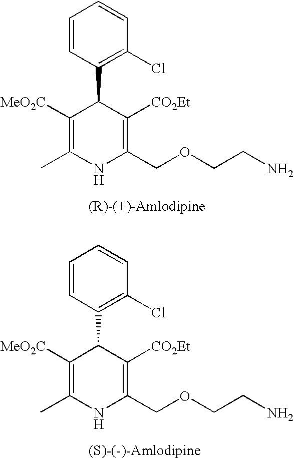

(R)-(+)-amlodipine and (S)-(−)-amlodipine have pharmacological functions different from each other. For example, (R)-(+)-amlodipine, despite its lack of calcium-channel blocking activity, is a potent inhibitor of smooth muscle cell migration, which is useful for preventing arterosclerosis and restenosis. (S)-(−)-amlodipine has blood pressure lowering activity superior to (R)-(+)-amlodipine (See PCT Publication No. WO 1995/05822): its activity is 2 times higher than that of (R/S)-amlodipine (See J. Med. Chem. 1986, 29, 1696-1702).

Amlodipine in the form of a free base shows low stability. Therefore, it is preferably administrated in the form of a pharmaceutically acceptable acid addition salt. In this regard, various acid addition salts of (S)-(−)-amlodipine have been developed.

PCT Publication No. WO 2006/043148 discloses (S)-(−)-amlodipine besylate hemipentahydrate and (S)-(−)-amlodipine besylate dihydrate, but, does not mention the specific pharmacological, physical or chemical properties thereof.

Korean Patent Application Publication No. 2005-37498 discloses that (S)-(−)-amlodipine besylate dihydrate has improved water-solubility and high bioactivity. However, this salt has poor photostability when exposed to sunlight. Korean Patent No. 515294 discloses (S)-(−)-amlodipine nicotinate dihydrate having good effect on blood pressure lowering. However, this salt also has poor photostability when exposed to sunlight.

Korean Patent Application Publication No. 2005-61317 discloses (S)-(−)-amlodipine gentisate which has superior photostability over (S)-(−)-amlodipine besylate. However, this salt has poor water-solubility (its solubility in distilled water is about 1 mg/ml), which is not suitable for pharmaceutical use.

Accordingly, there is a need for developing a novel salt of (S)-(−)-amlodipine having improved photostability and solubility.

### SUMMARY OF THE INVENTION

It is a primary object of the present invention to provide a novel acid addition salt of (S)-(−)-amlodipine having enhanced photostability and solubility.

In accordance with one aspect of the present invention, there is provided an (S)-(−)-amlodipine camsylate of formula (I):

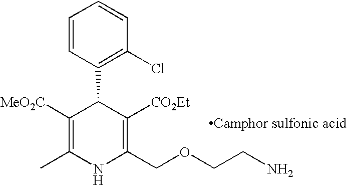

wherein, camphor sulfonic acid is (1S)-(+)-10-camphor sulfonic acid or (±)-10-camphor sulfonic acid.

The present invention also provides an (S)-(−)-amlodipine camsylate hydrate of formula (II):

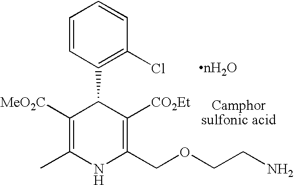

wherein, camphor sulfonic acid is (1S)-(+)-10-camphor sulfonic acid or (±)-10-camphor sulfonic acid; and n is 1 to 2.

The present invention further provides a pharmaceutical composition for treating cardiovascular diseases, comprising the (S)-(−)-amlodipine camsylate or the hydrate thereof as an active ingredient.

### BRIEF DESCRIPTION OF THE DRAWINGS

The above and other objects and features of the present invention will become apparent from the following description of the invention taken in conjunction with the following accompanying drawings, which respectively show:

FIG. 1: an X-ray diffraction scan of the inventive (S)-(−)-amlodipine (1S)-(+)-10-camsylate hydrate;

FIG. 2: an X-ray diffraction scan of the inventive (S)-(−)-amlodipine (1S)-(+)-10-camsylate anhydride;

FIG. 3: an X-ray diffraction scan of the inventive (S)-(−)-amlodipine (±)-10-camsylate hydrate; and

FIG. 4: a graph showing the time-dependent degradation of (S)-amlodipine salts when exposed to sunlight.

### DETAILED DESCRIPTION OF THE INVENTION

The inventive (S)-(−)-amlodipine camsylate can be prepared by (a) resolving amlodipine racemate into (S)-(−)-amlodipine free base and (b) reacting the (S)-(−)-amlodipine free base with camphor sulfonic acid in a solvent, as shown in Reaction Scheme 1.

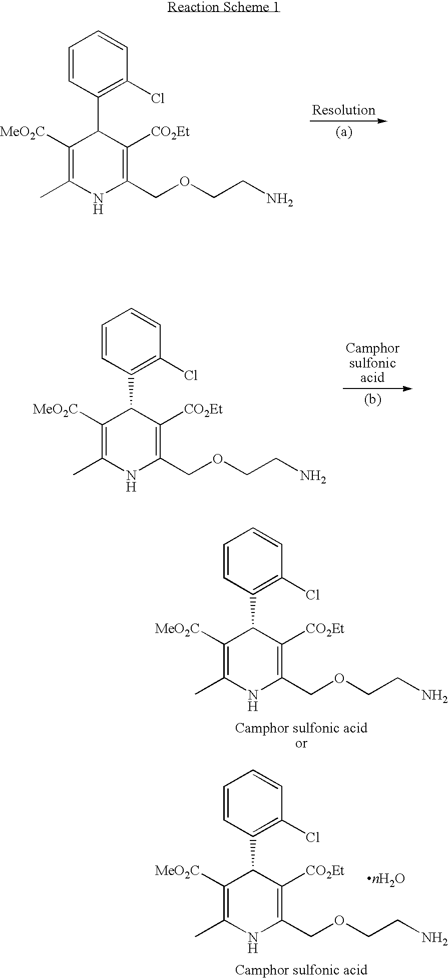

wherein, camphor sulfonic acid is (1S)-(+)-10-camphor sulfonic acid or (±)-10-camphor sulfonic acid; and n is 1 to 2.

In reaction scheme 1, step (a) may be conducted by the method disclosed in PCT Publication WO 95/25722, to obtain (S)-(−)-amlodipine free base having an optical purity of 99% (ee) or higher.

Step (b) may be conducted in a mixture of an organic solvent and water, or a mixture of a polar solvent and a non-polar solvent, to obtain (S)-(−)-amlodipine camsylate in a hydrate or anhydride form depending on the solvent used.

For example, when the reaction solvent is a mixture of water and an organic solvent miscible with water, e.g., methanol, ethanol, isopropanol, acetonitrile and acetone, preferably isopropanol, (S)-(−)-amlodipine camsylate is produced in a hydrate form in which one (S)-(−)-amlodipine camsylate molecule is coordinated to one to two H₂O molecules. In particular, (S)-(−)-amlodipine (1S)-(+)-10-camsylate hydrate has a moisture content of 4 to 6%, and (S)-(−)-amlodipine (±)-10-camsylate hydrate has a moisture content of 5 to 6%.

The mixture of an organic solvent and water may have a mix ratio of 1:1 to 1:30 (v/v), preferably 1:5 to 1:15 (v/v).

When the reaction solvent is a mixture of a polar solvent (e.g., methanol, ethanol, isopropanol, acetonitrile, acetone, diethyl ether, methyl t-butyl ether and a mixture thereof) and a non-polar solvent (e.g., hexane, heptane and a mixture thereof), (S)-(−)-amlodipine (1S)-(+)-10-camsylate anhydride is produced. Such anhydride form converts into a hydrate form when it absorbs moisture from the atmosphere.

In the present invention, the reaction solvent may be used in an amount of 5 to 50 ml, preferably 10 to 30 ml based on 1.0 g of (S)-(−)-amlodipine free base.

Also, step (b) may be conducted at a temperature of 0 to 50° C., preferably 10 to 30° C., for 2 to 24 hours.

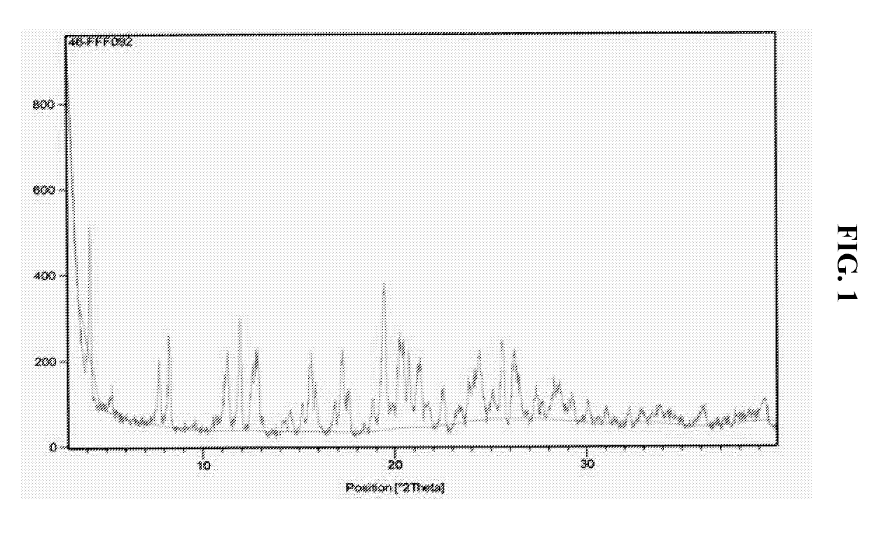

The inventive (S)-(−)-amlodipine-camsylate or a hydrate thereof thus prepared has a specific X-ray diffraction pattern which is different from those of the known (S)-(−)-amlodipine salts, as shown in FIGS. 1 to 3.

The inventive (S)-(−)-amlodipine-camsylate may be converted into an amorphous form by a conventional method such as solvent precipitation, freeze drying and spray drying.

Furthermore, the inventive (S)-(−)-amlodipine-camsylate or a hydrate thereof may be formulated together with a conventional antihypertensive agent (e.g., diuretic, ACE inhibitor, calcium channel blocker and angiotensin receptor blocker), as well as a conventional antihyperlipidemic agent (e.g., lovastatin, simvastatin, atorvastatin, rosurvastatin and fluvastatin).

Accordingly, the present invention provides a pharmaceutical composition for treating cardiovascular diseases, comprising the inventive (S)-(−)-amlodipine camsylate or a hydrate thereof as an active ingredient.

The pharmaceutical composition may be administered via various routes including oral and parenteral application, and formulated by using the conventional pharmaceutically acceptable diluents or excipients such as filler, extender, binder, wetting agents, disintegrants and surfactants.

The solid formulation for oral administration may be the form of a tablet, pill, powder, granule or capsule, which may comprise at least one excipient such as starch, sucrose, lactose and gelatin, and lubricant such as magnesium stearate and talc.

The liquid formulation for oral administration may be the form of a suspension, solution, emulsion or syrup, which may comprise a diluent such as water and liquid paraffin, and at least one excipient such as wetting agent, sweeter, flavoring agent and preservatives.

The formulation for parenteral administration may be the form of a sterile aqueous solution, non-aqueous solution, suspension, emulsion, freeze-dried product or suppository. The non-aqueous solution or suspension may comprise propylene glycol, polyethylene glycol, vegetable oil such as olive oil and injectable ester such as ethylolate. The suppository may be prepared by using a base such as witepsol, macrogol, Tween 61, cacao butter, laurin butter and glycerol gelatin.

A typical daily dose of the inventive (S)-(−)-amlodipine-camsylate or a hydrate thereof may range from about 1.0 to 5.0 mg/kg body weight, preferably 2.5 to 4.0 mg/kg body weight, and can be administered in a single dose or in divided doses.

The present invention will be described in further detail with reference to Examples. However, it should be understood that the present is not restricted by the specific Examples.

### Example

### Preparation 1: Preparation of S-(−)-amlodipine-hemi-D-tartrate-mono-dimethyl sulfoxide solvate

1.5 kg of (R/S)-amlodipine was dissolved in 7.5 l of dimethyl sulfoxide, to which a solution of 275.3 g of D-(−)-tartaric acid in 7.5 l of dimethyl sulfoxide was slowly added dropwise with stirring at room temperature. The resulting slurry was further stirred at room temperature for 12 hours, and the precipitated solid was filtered, washed with 6.0 l of dimethyl sulfoxide and 6.0 l of acetone, and dried under a warm air flow at 40° C. overnight, to obtain 771 g (yield: 37.4%) of the title compound in the form of a white solid.

Optical purity: 98.2% ee

### Preparation 2: Preparation of S-(−)-amlodipine Free Base

770.0 g of S-(−)-amlodipine-hemi-D-tartrate-mono-dimethyl sulfoxide solvate obtained in Preparation 1 was added to 7.7 l of dichloromethane, to which 8.6 l of 2N sodium hydroxide solution was slowly added dropwise, and the resultant was stirred at room temperature for 40 minutes. The organic layer was separated, washed with 7.7 l of water, dried over anhydrous sodium sulfate and filtered. Dichloromethane was removed under a reduced pressure, and 1.5 l of hexane was added to the oily residue, followed by evaporation of hexane to obtain a precipitate. To the precipitated white slurry, 9 l of hexane was slowly added, and the resultant was stirred at room temperature for 4 hours, filtered, washed with hexane, and dried under a warm air flow at 40° C., to obtain 525.8 g (yield: 93.9%) of the title compound in the form of a white solid.

Optical purity: 99.9% ee

### Example 1

### Preparation of S-(−)-amlodipine (1S)-(+)-10-camsylate hydrate

300 g of (S)-amlodipine free base obtained in Preparation 2 was added to a mixture of 900 mg of isopropanol and 900 mg of distilled water, and 170.4 g of (1S)-(+)-10-camphor sulfonic acid was added thereto, and the resulting mixture was warmed to obtain a homogeneous solution. To this solution, 30.0 g of activated carbon was added and stirred at room temperature for 1 hour. The mixture was then filtered through celite and washed with 300 ml of isopropanol and 300 ml of distilled water. 6.3 l of distilled water was slowely added to the filtrate, stirred at 20° C. for 3 hours, and the precipitated solid was filtered. The solid was washed with 600 ml of an isopropanol-water mixture (1:5, v/v), dried under a warm air flow at 40° C., to obtain 414 g (yield: 88.0%) of the title compound in the form of a white solid.

Optical purity: >99.9% ee

Moisture content: 4.4·4.6%

M.P.: 146.3˜150.5° C.

1H-NMR (300 MHz, CDCl₃) δ (ppm): 7.75(s, 4H), 7.45˜6.09(m, 4H, ArH), 5.39(s, 1H), 4.77(q, 2H), 4.03(m, 2H), 3.85(m, 2H), 3.58(s, 3H), 3.35(m, 2H), 3.05(q, 2H), 2.50˜2.20(m, 2H), 2.38(s, 3H), 2.10˜1.80(m, 3H), 1.75(m, 1H), 1.38(m, 1H), 1.15(t, 3H), 1.00(s, 3H), 0.80(s, 3H)

The crystalline state of the S-(−)-amlodipine (1S)-(+)-10-camsylate hydrate obtained was analyzed by X-ray diffraction spectroscopy (FIG. 1). The observed main peaks at characteristic diffraction angles are listed in Table 1.

TABLE 1

| 2θ | d | I/Iₒ |
|---|---|---|
| 4.2 | 21.2 | 100 |
| 7.8 | 11.4 | 45.9 |
| 8.3 | 10.7 | 66.3 |
| 11.3 | 7.8 | 53.3 |
| 11.9 | 7.4 | 80.3 |
| 12.5 | 7.1 | 36.3 |
| 12.9 | 6.9 | 46.7 |
| 16.7 | 5.7 | 54.6 |
| 17.3 | 5.1 | 51.9 |
| 17.6 | 5.0 | 31.4 |
| 19.5 | 4.6 | 87.1 |
| 20.2 | 4.4 | 62.7 |
| 20.4 | 4.3 | 55.2 |
| 20.7 | 4.3 | 57.2 |
| 21.3 | 4.2 | 44.9 |
| 24.4 | 3.7 | 48.0 |
| 25.6 | 3.5 | 53.5 |
| 26.2 | 3.4 | 46.9 |

2θ: Diffraction angle
d: Interplanar spacing
I/I₀ (%): Relative intensity of peak

### Example 2

### Preparation of S-(−)-amlodipine (1S)-(+)-10-camsylate anhydride

5 g of (S)-amlodipine free base obtained in Preparation 2 was added to 25 ml of isopropanol, in which 2.85 g of (1S)-(+)-10-camphor sulfonic acid was dissolved. To the resulting solution, 99 ml of methyl t-butyl ether (MTBE) and 2 ml of hexane were added and stirred at room temperature for 2 hours. The resulting solid was filtered under a nitrogen atmosphere and dried in vacuo, to obtain 6.4 g (yield: 81.5%) of the title compound in the form of a white solid.

Optical purity: >99.9% ee

Moisture content: 0.3%

M.P.: 145.5˜149.4° C.

¹H-NMR data was the same as that in Example 1.

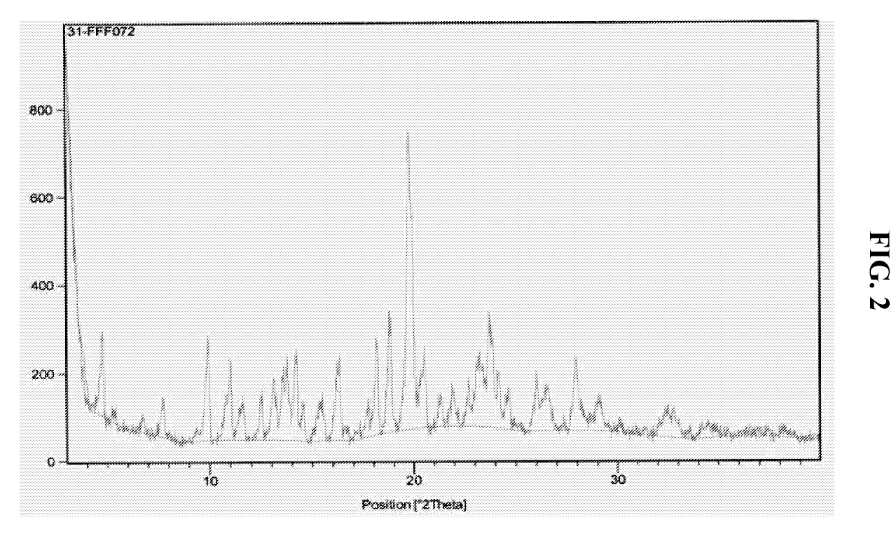

The crystalline state of the S-(−)-amlodipine (1S)-(+)-10-camsylate anhydride obtained was analyzed by X-ray diffraction spectroscopy (FIG. 2). The observed main peaks at characteristic diffraction angles are listed in Table 2.

TABLE 2

| 2θ | d | I/Iₒ |
|---|---|---|
| 4.8 | 18.6 | 28.0 |
| 10.0 | 8.9 | 35.5 |
| 11.0 | 8.0 | 27.3 |
| 13.8 | 6.4 | 30.0 |
| 14.3 | 6.2 | 25.8 |
| 16.4 | 5.4 | 26.9 |
| 18.2 | 4.9 | 30.9 |
| 18.8 | 4.7 | 39.2 |
| 19.8 | 4.5 | 100 |
| 20.0 | 4.5 | 67.2 |
| 20.5 | 4.3 | 27.6 |
| 23.7 | 3.8 | 36.1 |

2θ: Diffraction angle
d: Interplanar spacing
I/I₀ (%): Relative intensity of peak

### Example 3

### Preparation of S-(−)-amlodipine (±)-10-camsylate hydrate

10 g of (S)-amlodipine free base obtained in Preparation 2 was added to 20 ml of isopropanol, in which 5.68 g of (±)-camphor sulfonic acid was completely dissolved. Thereto, 200 ml of distilled water was slowly added dropwise. The resulting solution was stirred at room temperature for 3 hours and then at 15° C. for 2 hours, and the precipitated solid was filtered. The solid was washed with 25 ml of an isopropanol-water mixture (1:10, v/v), dried under a warm air flow at 40° C., to obtain 13.7 g (yield: 87.4%) of the title compound in the form of a white solid.

Optical purity: >99.9% ee

Moisture content: 5.4%

10 M.P.: 140.2˜142.6° C.

¹H-NM data was the same as that in Example 1.

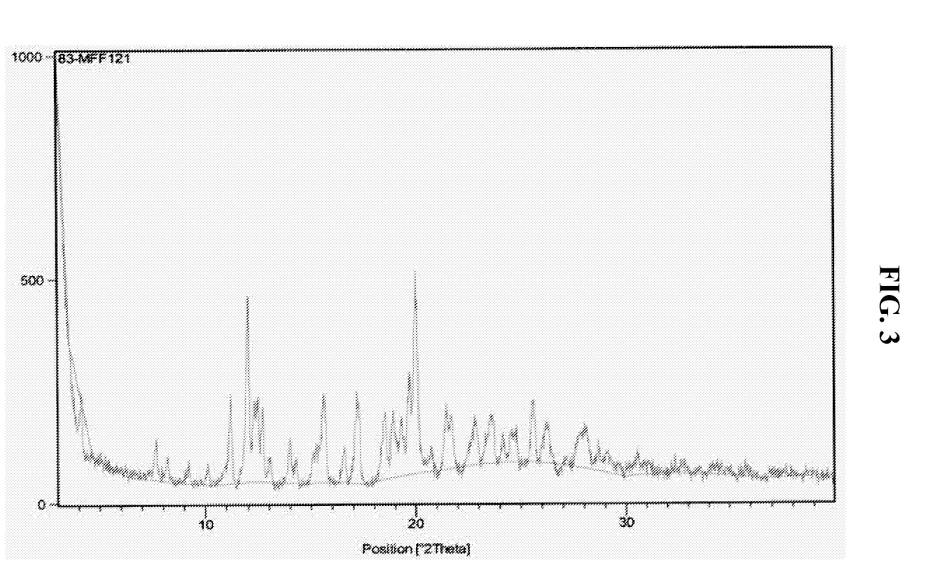

The crystalline state of the S-(−)-amlodipine (±)-10-camsylate hyrate obtained was analyzed by X-ray diffraction spectroscopy (FIG. 3). The observed main peaks at characteristic diffraction angles are listed in Table 3.

TABLE 3

| 2θ | d | I/Iₒ |
|---|---|---|
| 3.1 | 28.6 | 100 |
| 4.7 | 19.0 | 32.5 |
| 5.5 | 16.2 | 76.6 |
| 9.3 | 9.6 | 79.7 |
| 11.4 | 7.8 | 61.0 |
| 12.9 | 6.9 | 68.1 |
| 13.0 | 6.8 | 46.1 |
| 15.2 | 5.8 | 44.6 |
| 15.7 | 5.6 | 48.2 |
| 16.3 | 5.5 | 50.8 |
| 17.4 | 5.1 | 43.3 |
| 19.0 | 4.7 | 69.4 |
| 20.0 | 4.4 | 63.9 |
| 20.2 | 4.4 | 47.3 |
| 21.0 | 4.2 | 41.1 |
| 25.8 | 3.5 | 68.9 |

2θ: Diffraction angle
d: Interplanar spacing
I/I₀ (%): Relative intensity of peak

### Reference Example 1

### Preparation of S-(−)-amlodipine (R)-camsylate

10 g of (S)-amlodipine free base obtained in Preparation 2 and 5.68 g of (R)-camphor sulfonic acid were dissolved in 20 ml of isopropanol, to which 200 ml of distilled water was slowly added dropwise. The resulting solution was stirred at room temperature overnight, cooled to 15° C., and further stirred for 1 hour. The precipitated solid was filtered, washed with 25 ml of an isopropanol-water mixture (1:10, v/v), and dried under a warm air flow at 40° C., to obtain 9.77 g (yield: 62.3%) of the title compound in the form of a white solid.

Optical purity: >99.9% ee

Moisture content: 3.2%

### Experimental Example 1

### Photostability Test

A pharmaceutical formulation comprising an active ingredient must meet the required stability against humidity, temperature and light. In case of a drug for treating cardiovascular diseases such as hypertension, in particular, its photostability is important since it is generally prescribed together with another drugs for long-term medication in a paper-sealed state, which is usually exposed to light over a long period of time. Accordingly, the photostability of the (S)-(−)-amlodipine salts is very important.

In this regard, the photostabilities of the (S)-(−)-amlodipine salts obtained in Examples 1 to 3 and Reference Example 1 were measured and compared with those of the known (S)-(−)-amlodipine besylate (PCT Publication No. WO 2006/043148) and (s)-(−)-amlodipine nicotinate dihyrate (Korean Patent No.515294).

100 mg each of the above-mentioned 6 salts was respectively placed in 6 test tubes to prepare a total of 36 samples (6 samples per salt), and they were exposed to sunlight for 36 hours. Then, samples of each salt were taken at 6 hour-intervals and stored in a cool and dark place. After 36 hours, each sample was diluted with a 20 mM ammonium acetate buffer solution (pH=5.0)—acetonitrile mixture (1:1, v/v) and analyzed by HPLC under the following conditions:
Column: Symmetry C8 (4.6 mm×100 mm, 3.5 μm, Water, US)
Eluent: 1 l of a solution of 7 g perchloric acid monohydrate and 1.74 g potassium monohydrogen phosphate in purified water, which adjusted to pH 2.8 by the addition of phosphoric acid.

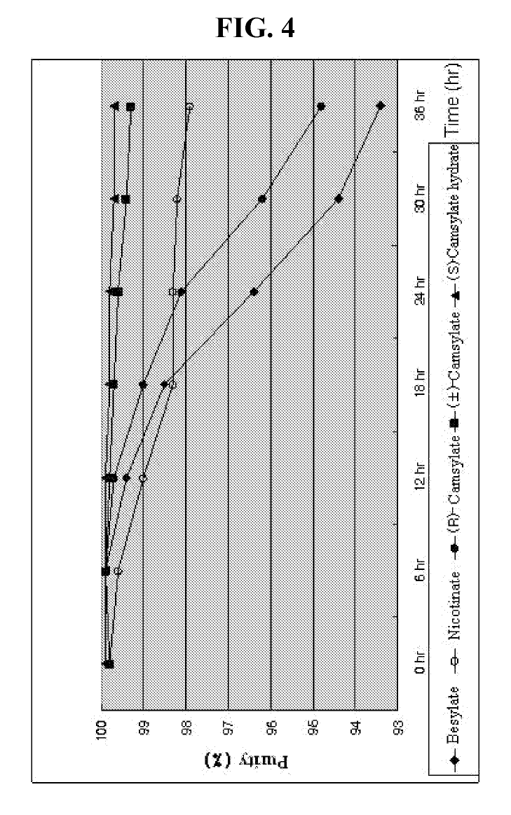

The results are shown in FIG. 4 and Table 4.

TABLE 4

| | Purity (area %) | | | | | | |
|---|---|---|---|---|---|---|---|
| (S)-(−)-amlodipine salt | Initial | 6 hours | 12 hours | 18 hours | 24 hours | 30 hours | 36 hours |
| Besylate | 99.8 | 99.9 | 99.4 | 98.5 | 96.4 | 94.4 | 93.4 |
| Nicotinate dehydrate | 99.8 | 99.6 | 99.0 | 98.3 | 98.3 | 98.2 | 97.9 |
| (R)-Camsylate | 99.8 | 99.9 | 99.7 | 99.0 | 98.1 | 96.2 | 94.8 |
| of Ref. Ex. 1 | | | | | | | |
| (±)-10-Camsylate | 99.8 | 99.9 | 99.8 | 99.7 | 99.6 | 99.4 | 99.3 |
| hydrate of Ex. 3 | | | | | | | |
| (1S)-(+)-10-Camsylate | 99.9 | 99.9 | 99.9 | 99.9 | 99.8 | 99.7 | 99.7 |
| anhydride of Ex. 2 | | | | | | | |
| (1S)-(+)-10-Camsylate | 99.9 | 99.9 | 99.9 | 99.8 | 99.8 | 99.7 | 99.7 |
| hydrate of Ex. 1 | | | | | | | |

As shown in FIG. 4 and Table 4, (S)-(−)-amlodipine (±)-10-camsylate hydrate as well as (S)-(−)-amlodipine (1S)-(+)-10-camsylate hydrate or anhydride of the present invention are highly stable even when exposed to sunlight for 36 hours. In particular, (1S)-(+)-10-camsylate salt exhibits superior photostability over (±)-10-camsylate salt. However, (S)-(−)-amlodipine (R)-camsylate has undergone about 5% degradation, and the known (S)-(−)-amlodipine besylate and (S)-(−)-amlodipine nicotinate dihydrate, about 7% and 2% degradation, respectively, after 36 hours.

Also, (S)-(−)-amlodipine besylate and (S)-(−)-amlodipine (R)-camsylate have undergone a color change of their surface from off-white to brown, and they becomes partially melted.

The above results suggest that the inventive (S)-(−)-amlodipine camsylate or a hydrate thereof has improved photostability as compared with the known (S)-(−)-amlodipine besylate and (S)-(−)-amlodipine nicotinate dihydrate.

### Experimental Example 2

### Solubility Test

A pharmaceutically acceptable active ingredient preferably has a solubility in water of not less than 1 mg/ml at pH 1 to 7.5, particularly at the blood pH value of about 7.4. Accordingly, the solubilities and pH value at the saturation points of the amlodipine camsylate salts obtained Examples 1 and 3 and Reference Example 1 were measured and compared with those of amlodipine besylate (Korean Patent Publication No. 1995-7228), amlodipine gentisate (Korean Patent Application Publication No. 2005-61317) and crystalline amlodipine camsylate (WO 2002/079158 A1). The measurement was performed according to the procedure described in Korean Pharmacopoeia which comprises the steps of dissolving each compound in distilled water to saturation, analyzing the saturated solution with liquid chromatography, and measuring the dissolved amount of each compound based on the amount of amlodipine free base. The results are shown in Table 5.

TABLE 5

| | 12.5 | 25.0 | 50.0 | |
|---|---|---|---|---|
| (S)-(−)-amlodipine salt | mg/ml | mg/ml | mg/ml | Aver. |
| Besylate | 2.63 | 2.63 | 2.61 | 2.62 |
| Gentisate | 1.03 | 1.03 | 1.02 | 1.03 |
| (1S)-(+)-10-Camsylate hydrate | 2.72 | 2.73 | 2.73 | 2.72 |
| of Ex. 1 | | | | |
| (R)-Camsylate of Ref. Ex. 1 | 3.93 | 3.93 | 3.94 | 3.93 |
| (±)-10-camsylate hydrate | 3.42 | 3.51 | 3.67 | 3.54 |
| of Ex. 3 | | | | |
| Crystalline (R/S)-amlodipine | 1.02 | 1.04 | 1.02 | 1.03 |
| (S)-camsylate | | | | |

As shown in Table 5, the solubility of the inventive (S)-(−)-amlodipine camsylate is higher than that of amlodipine besylate, and in particular, it is 2.6 times higher than the known gentisate salt or crystalline amlodipine camsylate.

While the invention has been described with respect to the specific embodiments, it should be recognized that various modifications and changes may be made by those skilled in the art to the invention which also fall within the scope of the invention as defined as the appended claims.

1. An (S)-(−)-amlodipine camsylate of formula (I): 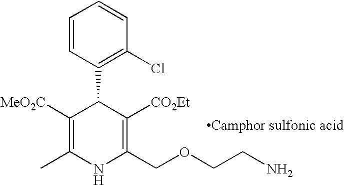
wherein, camphor sulfonic acid is (IS)-(+)-10-camphor sulfonic acid or (±)-10-camphor sulfonic acid.

2. The (S)-(−)-amlodipine camsylate of claim 1, wherein the camphor sulfonic acid is (1S)-(+)-10-camphor sulfonic acid and its X-ray powder diffraction spectrum shows major peaks at 2θ of 4.8±0.2, 10.0±0.2, 11.0±0.2, 13.8±0.2, 14.3±0.2, 16.4±0.2, 18.2±0.2, 18.8±0.2, 19.8±0.2, 20.0±0.2, 20.5±0.2 and 23.7±0.2.

3. The (S)-(−)-amlodipine camsylate of claim 1, which is of an amorphous form.

4. An (S)-(−)-amlodipine camsylate hydrate of formula (II): 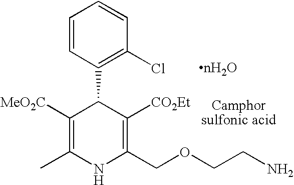
wherein, camphor sulfonic acid is (1S)-(+)-10-camphor sulfonic acid or (±)-10-camphor sulfonic acid; and n is 1 to 2.

5. The (S)-(−)-amlodipine camsylate hydrate of claim 4, wherein the camphor sulfonic acid is (1S)-(+)-10-camphor sulfonic acid and its X-ray powder diffraction spectrum shows major peaks at 2θ of 4.2±0.2, 7.8±0.2, 8.3±0.2, 11.3±0.2, 11.9±0.2, 12.5±0.2, 12.9±0.2, 16.7±0.2, 17.3±0.2, 17.6±0.2, 19.5±0.2, 20.2±0.2, 20.4±0.2, 20.7±0.2, 21.3±0.2, 24.4±0.2, 25.6±0.2 and 26.2±0.2.

6. The (S)-(−)-amlodipine camsylate hydrate of claim 4, wherein the camphor sulfonic acid is (±)-1 0-camphor sulfonic acid and its X-ray powder diffraction spectrum shows major peaks at 2θ of 3.1±0.2, 4.7±0.2, 5.5±0.2, 9.3±0.2, 11.4±0.2, 12.9±0.2, 13.0±0.2, 15.2±0.2, 15.7±0.2, 16.3±0.2, 17.4±0.2, 19.0±0.2, 20.0±0.2, 20.2±0.2, 21.0±0.2 and 25.8±0.2.

7. A pharmaceutical composition for treating cardiovascular diseases comprising the (S)-(−)-amlodipine camsylate of claim 1.

8. The pharmaceutical composition of claim 7, wherein the cardiovascular disease is angina pectoris, hypertension or congestive cardioplegia.

9. A pharmaceutical composition for treating cardiovascular diseases comprising the (S)-(−)-amlodipine camsylate hydrate of claim 4 as an active ingredient.

10. The pharmaceutical composition of claim 9, wherein the cardiovascular disease is angina pectoris, hypertension or congestive cardioplegia.
